(19) United States
(12) Patent Application Publication
(10) Pub. No.: US 2026/0049976 A1
(43) Pub. Date: Feb. 19, 2026

(54) Ballistic microscopy (BaM): High-throughput cytoplasm spatio-temporal pico-sampling from live-single cells for omic studies using particle bombardment

(71) Applicant: The Board of Trustees of the Leland Stanford Junior University, Stanford, CA (US)

(72) Inventors: Manu Prakash, San Francisco, CA (US); Jijumon A S, Palo Alto, CA (US)

(21) Appl. No.: 19/103,333

(22) PCT Filed: Aug. 23, 2023
(86) PCT No.: PCT/US2023/030968

§ 371 Date: Feb. 12, 2025

Related U.S. Application Data
(60) Provisional application No. 63/400,284, filed on Aug. 23, 2022.

(51) Int. Cl. G01N 33/487; G01N 27/62; G01N 33/50; G01N 33/543

(52) U.S. Cl. CPC G01N 33/487; G01N 27/62; G01N 33/5005; G01N 33/543

(57) ABSTRACT

Ballistic microscopy—a completely new approach to “image” a cell utilizing particle bombardment, is described. These are ballistic micro and nano particles that travel through a cell at ballistic speed and capture a pico or femto-liter of cellular content and bring it out for analysis without harming the cell. This enables a new approach to omics-based imaging where millions of these particles are bombarded on cells with resolved space and time and captured to process using well known omics techniques including proteomics (mass spec) or sequencing—while keeping the spatial and temporal resolution. This work provides—for the first time—a way to resolve atomic details of live cells without any labels.

### FIELD OF THE INVENTION

This invention relates to sampling of cell contents for characterization.

### BACKGROUND

Cells are heterogenous with hundreds of thousands of molecular machines interacting with each other. How these machines work in a dynamic context of the cell remains largely unknown. Currently, state of the art atomic resolution imaging operates on label based approaches (see what we are looking for). Label free approaches like cryo-electron microscopy give us a single snapshot frozen in one state. Mass spectrometry can decipher molecular identities of unknown components, but is also limited to a single snapshot destroying the sample of interest and provides limited spatial resolution. Accordingly, it would be an advance in the art to provide improved sampling of cell contents.

### SUMMARY

We present BaM: Ballistic microscopy—a completely new approach to “image” a cell utilizing particle bombardment. These are ballistic micro and nano particles that travel through a cell at ballistic speed and capture a pico or femto-liter of cellular content and bring it out for analysis without harming the cell. We present a new approach to omics-based imaging where millions of these particles are bombarded on cells with resolved space and time and captured to process using well known omics techniques including proteomics (mass spec) or sequencing—while keeping the spatial and temporal resolution. This work provides—for the first time—a way to resolve atomic details of live cells without any labels.

These unmet needs described above open completely new avenues in research and medical science with unprecedented detail of observation of cellular heterogeneity in real time.

Significant advantages are provided:
1) Currently no technology exists that allows us to see spatio-temporal heterogeneity of components inside a cell—in a live cell assay.
2) The approach presented here changes our perspective of how we utilize mass spectroscopy, cry-electron microscopy and single cell sequencing. To be able to bring some of the most widely used and powerful techniques in biology currently—in a dynamic context of live cells opens new doors for understanding living systems at atomic resolution.
3) It should be possible to obtain atomic scale information of unknown proteins and exact location/time they appear in a living single cell. This is an untargeted approach capable of identifying and specifically pin-pointing unknown analytes and labeling them in both space and time within a volume of a single cell.
4) The technique is agnostic to cell type and works for cells with harshest cell walls (plant cells, tissues or organisms) or various environmental barriers—thus making it amenable to all cell and tissue types. The thickness of the tissue is not a key hurdle since by increasing the velocity of the particles, penetration depth can be varied from 10 microns to 500 microns.

### BRIEF DESCRIPTION OF THE DRAWINGS

FIGS. 1A-F show several concepts relating to embodiments of the invention.

FIG. 2A schematically shows some relevant particle parameters.

FIG. 2B shows an exemplary experimental setup.

FIGS. 2C-D show proof of concept experimental results.

FIG. 3 shows an exemplary arrangement having in-line characterization of the nanoparticles.

FIG. 4 shows a laser process for generating ballistic nanoparticles.

FIG. 5 schematically shows a vision and some applications of the present approach.

FIG. 6 shows two exemplary particle bombardment options.

FIGS. 7A-B show two exemplary particle capture options.

### DETAILED DESCRIPTION

BaM—short for Ballistic microscopy—is a completely new approach to “image” a cell utilizing particle bombardment. Instead of imaging a cell with photons or electrons; we “image” cells with high speed ballistic nanoparticles, reaching 3.5 km/sec. Traveling through cells at speeds ranging from 200 m/sec to 3,500 m/sec; nanoparticles (10 nm to 500 nm) can capture atto to zepto-liter of cellular content and bring it out of the cell in a direct-to-mass-spec, cryo-EM (electron microscope) or sequencing pipeline—without harming the cell. The particle takes ˜100 picosecond to pass through the cell—providing unprecedented atomic scale resolution (from mass-spec, cryo-EM or sequencing) at a 100-picosecond temporal resolution—opening a whole world of live cell imaging at atomic resolution.

Although we have a long list of molecular techniques current experiments fail to capture the details of cellular complexity. A critical missing link is lack of high resolution “dynamic imaging” techniques. Three state of the art techniques have been a work horse of our current understanding of biological systems in the last 3 decades—namely mass spectrometry (and variations), cryo-electron microscopy in its current form and single cell sequencing and associated omics techniques. Each of these techniques brings to the table an unprecedented resolution in studying molecular components—revealing untargeted, unknown analytes in a highly complex mixture. But all three of these techniques suffer from a major drawback—each of them is a destructive approach to imaging biological systems, and hence they never been utilized in a context of a living cell. Furthermore, mass-spec and sequencing have a further drawback of almost no spatial resolution when it comes to knowing where in the cell exactly the particular molecule came from; while cryo-imaging also fails to perform well in traditional cells without significant milling.

Our ballistic microscopy framework can “bring to life” a number of high-resolution techniques including mass-spectrometry, cryo-imaging and next generation sequencing to high spatial and temporal resolution in live dynamic cells. Our approach converts well-adopted and established techniques that were previously static into a dynamic framework. We do this by breaking the problem into two components (1) high-throughput spatial and temporal sampling (from 10⁻²¹ m³ to 10⁻²⁴ m³) of live cells and tissues without damage (2) an in-line pipeline from sample generation to analysis utilizing mass-spectrometry, cryo-EM or nucleic acid sequences. This combination makes previous techniques that could not be used at high spatial or temporal resolution—to truly enable dynamic imaging at atomic resolution.

The idea—in principle—is very simple. Instead of imaging with photons (light microscopy) or electrons (electron microscopy)—we consider an entirely new methodology of imaging with “nano-particles”.

First, via utilizing various well-established techniques in the literature—we make ballistic nanoparticles that travel through air when generated at speeds ranging from 200 m/sec all the way to 3.5 km/sec. Although these are unprecedented speeds, standard protocols including ballistics-based gene delivery platforms and more recently introduced technique of laser induced projectile impact test (LIPIT) technique achieve these speeds routinely in laboratory setting. We have built and demonstrated a setup that allows us to achieve 500 m/sec in our current preliminary results.

Second, we use this ballistic spray of thousands to millions of particles traveling through an aperture (pin hole) and “image” either cells, tissue slices, organoids or whole organisms. Due to very high kinetic energy associated with the individual nanoparticles; these small length scales still maintain themselves in high Reynolds number regime when they travel through the fluid media—and ultimately through the cell from top to bottom. The transit time through the cell at these speeds can be as low as a few hundred picoseconds. In doing so, we hypothesized and later confirmed that cytoplasmic material from the cell is captured in atto-liter to zepto-liter volumes and carried via the nano-particle outside the cell. Crucially, this process does not damage the cell due to incredible high velocity and such small size of the nano-particle. Lastly, the nanoparticle acts as a carrier to biological material captured at a specific location and time in the cell. These nanoparticles can either be stopped and frozen in its path in a gel matrix for further sequential analysis or directly in-line introduced into a mass-spec or electron microscopy or sequencing pipeline for real-time analysis. With such a short transit time—a particle can be fired through the cell every micro-second and hence a dynamic high-speed movie of the processes at play can be observed in an untargeted approach.

### 1) MATHEMATICAL FRAMEWORK

As is well known via Stokes laws, the low Reynolds number regime dominated by viscosity is dramatically different from high Reynolds number regime dominated by inertia. Here Reynolds number Re is given by ratio of inertial to viscous forces (Re=ρ_{f}vD/η). For the parameters discussed above—it is clear that this parameter is sensitive to the product of velocity and diameter (all other parameters being equal). So, for a 1 nm particle traveling at 1 km/sec—it is indeed a low Reynolds number problem while for 500 nm particle traveling at same velocity—it is an intermediate Re number phenomena. This is critical since Re number allows us to estimate the associated drag of a particle traveling either through air or water, akin to a gel such as a cell or hydrogel base.

We compute the drag on ballistic nano-particles traveling at a velocity range of 100 m/sec to 3 km/sec to be given by Drag coefficient (C_{d})

$${C}_{d} = \frac{24}{R e} ( 1 - H ( R e - 0.2017245 ) ) + ( \frac{21.12}{R e} + \frac{6.3}{\sqrt{R e}} + 0.25 ) H ( R e - 0.2017245 )$$

and associated de-acceleration to be given by

$$a = \frac{d v}{dt} = - \frac{3}{8} {C}_{d} ( R e ) \frac{{ρ}_{f}}{{ρ}_{p}} \frac{{v}^{2}}{R} .$$

Thus we have an explicit (albeit complex) expression of how a particle slows down in a medium—even at such ballistic speeds. By looking at the parameter space, we find that 100 nm particles traveling above the speed of sound will easily penetrate 10 microns. While at the same speed, a 500 nm particle would travel and penetrate as deep as 80 microns. Thus as long as the sample is thinner than this critical “penetration depth”—the particles pass through and can either be captured in the gel for later analysis or directly fed into an in-process for mass spectroscopy or sequencing.

### 2) NANOPARTICLE ENGINEERING

BaM depends on two key aspects—ballistic motion of nanoparticles through the cell without damage to cellular processes while simultaneously capturing a small atto-liter volume of cytoplasm for interrogation outside the cell.

Thus a particle, say 10 nm in size interacts with cellular cytoplasm for 100 nano-second (transit time) or smaller as it passes through the cell. Beyond the kinetic properties of this particle (as detailed above in Mathematical framework), surface properties and shape/size also determine the amount and type of cargo the ballistic projective is capable of pulling out of a cell. Here we explore the range of properties we intend to test and validate in this framework. Relevant particle parameters include particle size, shape and surface functionalization.

### 3) PROJECTILE MOTION GENERATION TECHNIQUES

### 3a) Traditional Ballistic/Gene Gun-Based Approach:

The idea of using ballistic nano-particles has been previously explored in biology—in the context of gene delivery. This common framework allows to bring particles that are pre-coated with biomolecules to penetrate cells with various barriers. We take this existing method—and flip it on its head. Here we propose to use this approach—not to bring things into the cell; but to actually bring things out of the cell. This allows us to prepare atto-to-zepto liter volume samples from a living cell or organism in a controlled manner. The equipment for ballistic gene gun operates on high pressure helium gas—up to 3000 psi. We have already developed this system which is already generating particles at the speed of 500 m/second. This is a good system to start—but for more precise localization of the particles—we expect laser induced techniques to be preferable.

### 3b) Laser Induced Projectile Impact Test (LIPIT):

Instead of broad wide field of particle projectiles—over the last decade a new class of laser induced projectile impact methods have been developed with fastest reported projectile velocities of 3.5 km/sec. This allows us a very wide range of velocity range in a compact form factor with single particle acceleration protocol. Since the laser can be positioned at any specific spot—a cell or tissue region specific probing can be done that allows for sampling exactly where it is needed as per experiment design. LIPIT has an added advantage of being able to use the laser as a trigger for another interferometer enabling direct time of flight measurement.

### 4) ESTIMATION AND MEASUREMENT OF CELLULAR DAMAGE

With velocities as high as 1 km/second, how do we explore the damage that such a particle might do to the cell. The first associated evidence of limited damage comes from wide use of gene gun for particle and gene delivery—roughly at a similar strain rate. We have further performed live/dead assays for Hela cells and already seen minimal cell damage during translocation of the cells via ballistic probes. Next we intend to optimize the size of the nanoparticle probe and perform a systematic screen for cell viability. From our first set of preliminary experiments, we expect to see 100% viability in cells under repeated ballistic probing. We will further perform theoretical calculations of energy deposited in the cell during a 100 pico second transit—using total translocation kinetic energy loss as the particle passes through the cell as an estimate.

### 5) FURTHER ASPECTS AND APPLICATIONS OF THIS WORK INCLUDE THE FOLLOWING

5a) Applicability on a wide variety of sample types including: (a) single eukaryotic cells including non-model systems that have been previously difficult to probe at molecular resolution with no known labels or capacity to penetrate a cell without damage (b) live tissue slices such as brain slices—to observe molecular processes in thick samples impossible to penetrate with light (c) whole animal live imaging bringing already established destructive techniques to be operated in a temporal context in live animals.
5b) Versatility by bringing a number of well-established atomic resolution techniques into the realm of live imaging: In order to do so, we expect that BaM is compatible with high resolution mass spectrometry, single particle cryo-EM imaging and single particle sequencing techniques. This is the first time all of these techniques can be brought into spatially and temporally controlled “imaging” paradigm with.
5c) High temporal resolution: Nano-particles shot at a biological sample have a transit time of less than 100 pico second inside a cell of thickness 10 microns. Thus the technique provides a tremendous temporal resolution—effectively capable of capturing a sample from the same spot every 1 micro-second. Furthermore, both in-line real time techniques and particle capture techniques we present here allow us to resolve particles that are shot so close to each other via modulating particle ballistic velocity ever so slightly. We expect that temporal resolution of this technique can reach 1 microsecond.
5d) Specificity of capturing membrane associated proteins vs bulk cytoplasm: BaM relies on interaction of nano-particles which act as capture probes for cytoplasmic materials. With a wide range of choices available in size, shape and surface characterization of the nano-particles; we intend to decipher what class of particle type will show affinity for membrane based capture vs bulk cytoplasm. From a theoretical framework; it is feasible to also capture a double membrane coated particle if surface affinity is too high. By systematically varying particle type, shape and coat; we expect to identify and screen particles optimal for specific function.
5e) A mathematical framework for defining key parameters to optimize BaM: Every scientific techniques has a series of sweet spots when it performs its best. By using a combined experimental and theoretical approach—we intend to establish the parameter range of BaM. We will demonstrate that starting with optimal particle size and type to optimal velocity that allows us to capture just the right amount of material for sequential in-line technique.

### 6) EXEMPLARY EMBODIMENTS

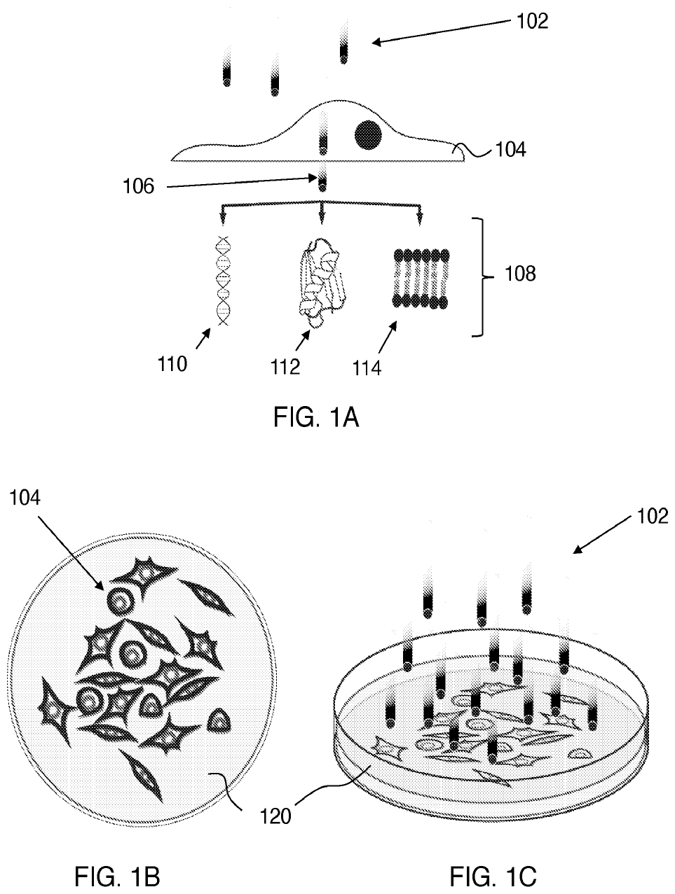

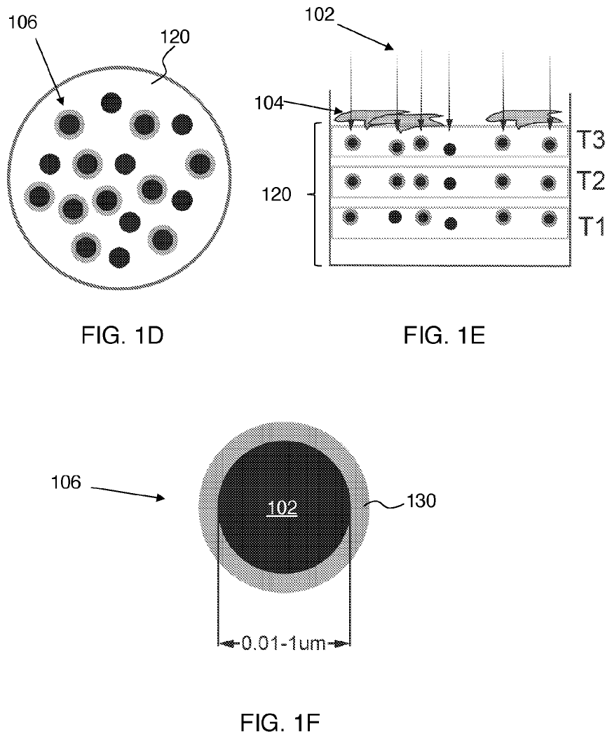

FIGS. 1A-F show concept figures for BaM. As shown on FIG. 1A, we consider the of use ballistic nano-particles 102 traveling at nearly kilometer/sec that traverse through cells 104 in less than a 100 pico-second—as an imaging probe; bringing mass-spectrometry, cryo-EM and sequencing based approaches to live cell imaging. Particles 106 that have passed through cell 104 can have cell contents on their surface which can be characterized 108 in various ways (e.g., nucleic acid analysis 110, proteomic analysis 112, and lipid cell membrane studies 114). The method is applicable for a broad range of single cells, tissue slices and even flat organisms—including non-model systems with untargeted, unknown analytes of interest.

FIGS. 1B-1C shows top and isometric views, respectively, of cells 104 attached on a hydrogel matrix 120. FIGS. 1D and 1E are top and side views, respectively, of captured nanoparticles 106 in the hydrogel matrix 120. The capture process both preserves the atto-to-zepto liter volume samples and registers them with respect to exact location in the cell. FIG. 1E shows that by varying ballistic velocity, particles fired at different times can be captured and isolated—thus recording time. FIG. 1F shows an enlarged view of a single nanoparticle 102 covered by cell contents 130 to provide a particle 106 suitable for further characterization. Thus FIGS. 1D-E show particles that have cell contents on them (gray edge region) and particles that don't have cell contents on them (no gray edge region).

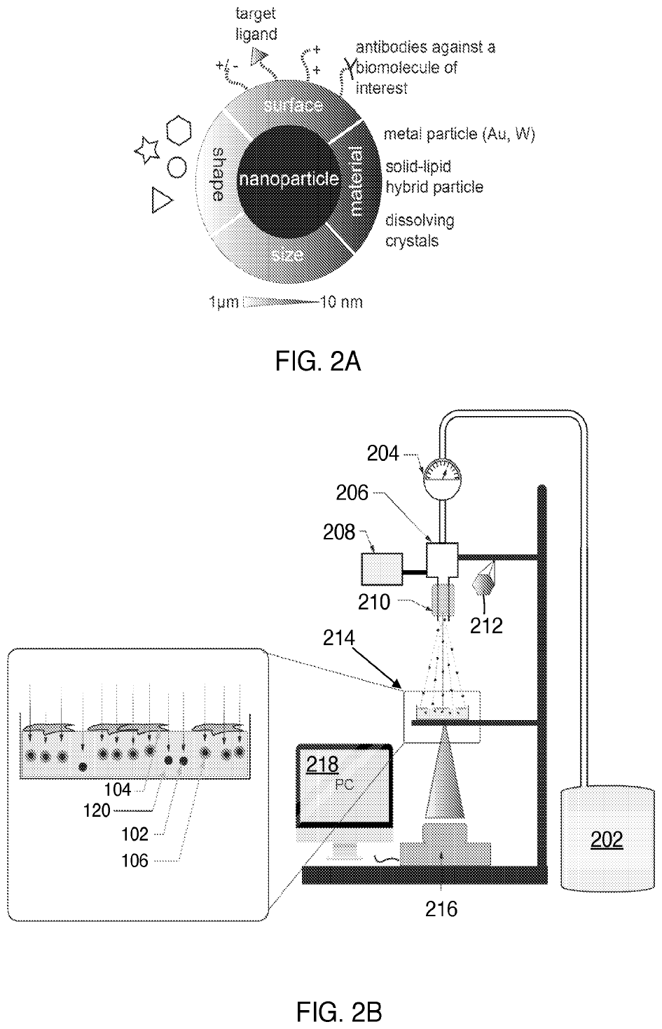

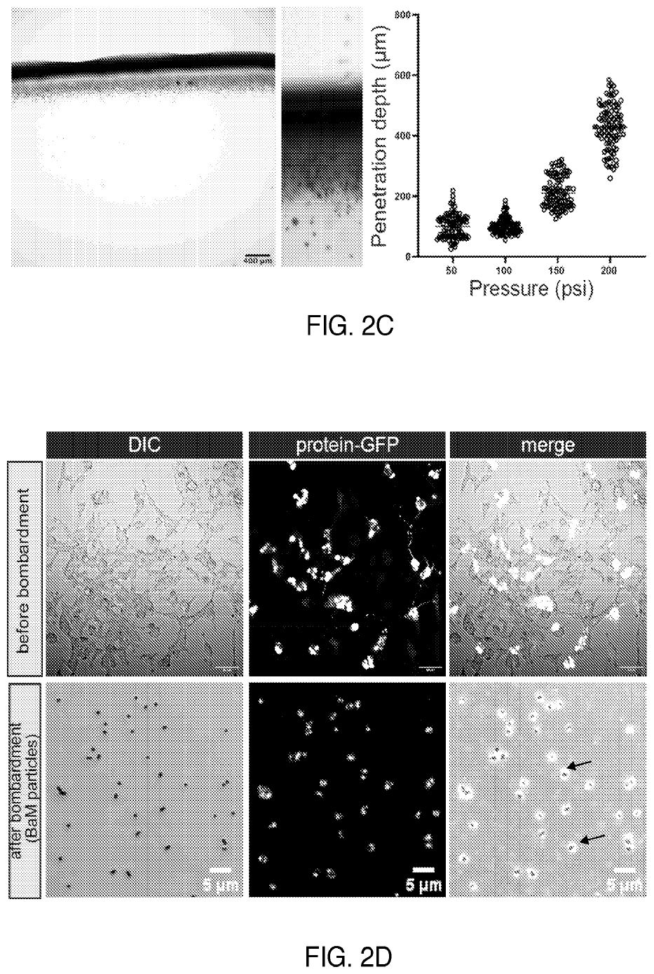

FIG. 2A schematically shows some exemplary relevant particle parameters. Particle shape, size, surface and material properties determine the volume and type of material captured by a BaM particle during a transit through a live cell. Suitable nanoparticle compositions include but are not limited to: gold, tungsten and iron. Suitable nanoparticle shapes include but are not limited to: pyramids, spheres, rods and stars. Suitable nanoparticle surface functionalization binding types include but are not limited to: oligo-DNA binding, antibody-antigen binding, biotin binding, peptide binding, nickel-nitrilotriacetic acid binding, polyethylene glycol binding, and click chemistry binding. Although it is expected that nanoparticle parameters are preferably tailored to optimize specific applications, practice of the invention does not depend critically on nanoparticle parameters.

FIG. 2B is a schematic of an exemplary experimental setup. Here 202 is a high-pressure helium tank (e.g., 2600 psi), 204 is a high-pressure gauge, 206 is a solenoid valve, 208 is a control circuit, 210 is a convergent-divergent nozzle, 212 is an illumination source, 214 is a hydrogel matrix having the cells of interest on its surface as described above, 216 is a high-speed camera for fluorescence microscopy and 218 is a PC for system control.

FIG. 2C shows images (on the left) and quantitative results (right) showing penetration depth of ballistic nanoparticles in the hydrogel. Tests in hydrogels demonstrate that 1 micron particles are capable of penetrating as high as 600 microns inside the gel—with only a pulse from 200 psi (total system capacity 3600 psi).

FIG. 2D shows experimental evidence of collection of cell contents from cells using ballistic nanoparticles. Upper panel shows the microscopy images of live human cells expressing proteins tagged with a green fluorescent protein (GFP) before bombardment. Lower panel shows the microscopy images of penetrated ballistic nanoparticles having the GFP fluorescence signal after bombardment (see black arrows).

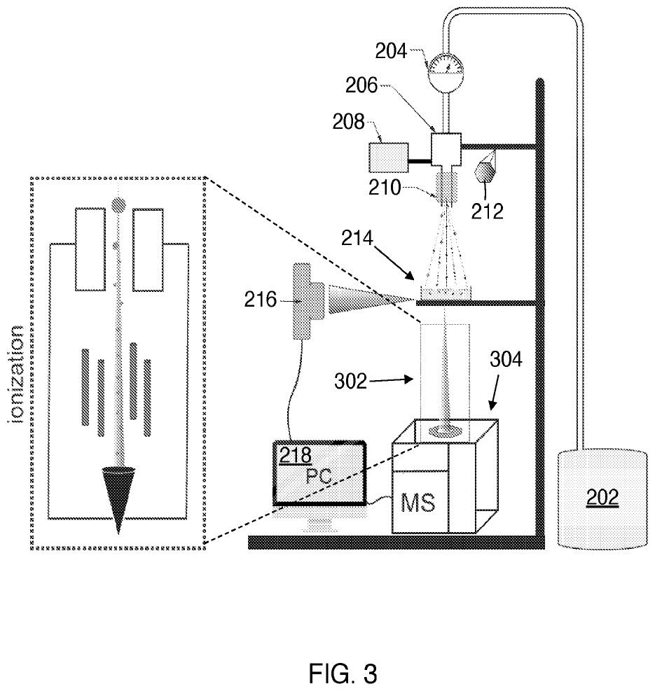

FIG. 3 shows a variation of the setup of FIG. 2B where the nanoparticles pass directly into an in-line mass spectrometry (MS) setup having an ionization region 302 and a MS instrument recorder 304.

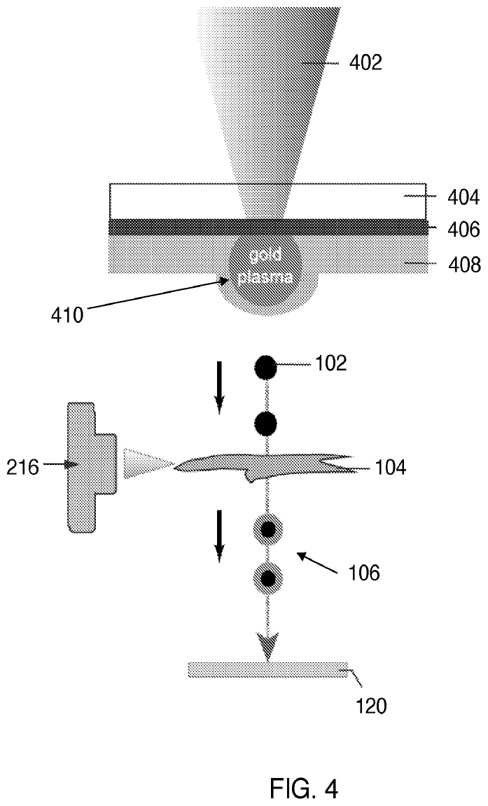

FIG. 4 schematically shows Laser Induced Particle Impact Testing (LIPIT) particle ejection. Here 402 is the laser pulse, 404 is a coverslip, 406 is a gold layer and 408 is a PDMS (Polydimethylsiloxane) layer. In operation, exposure of gold layer 406 to laser pulse 402 leads to formation of a gold plasma 410 and subsequent high-speed ejection of a nanoparticle 102 which passes through cell 104 to be characterized as described above. This approach has been used to demonstrate on-demand nanoparticle generation at a velocity of 3.5 km/sec at a localized spot of 100 nm.

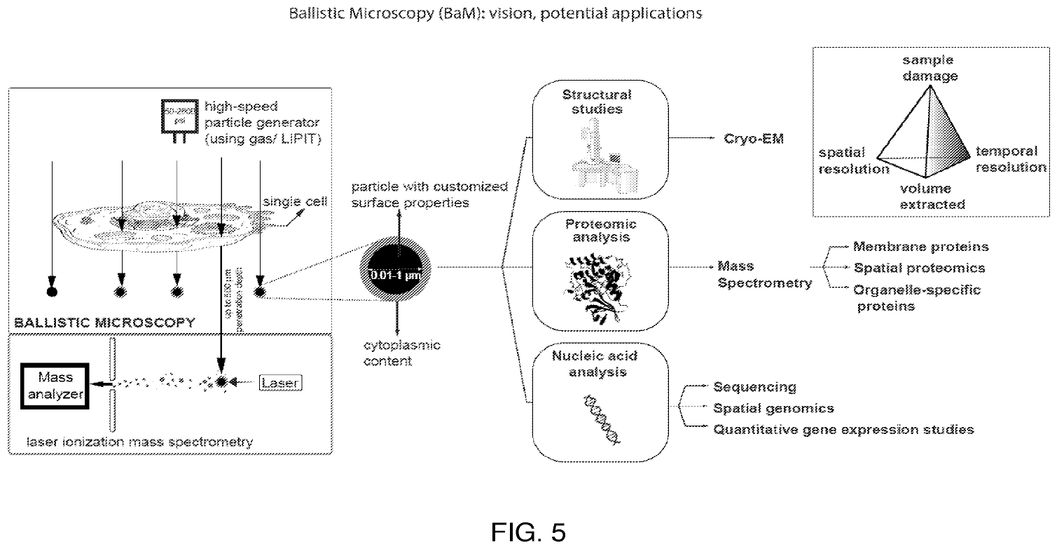

FIG. 5B schematically shows a vision and possible applications of BaM. We expect it to be feasible to couple BaM to current state of the art techniques including structural studies using Cryo-EM, proteomic analysis using techniques such as mass spectrometry, and nucleic acid analysis such as sequencing and spatial genomics. BaM brings all the above techniques into the realm of “dynamic imaging”. On the right of this figure, four constraints of BaM—including spatial resolution, temporal resolution, volume of cytoplasm or membrane captured and sample damage are considered. We expect to be able to optimize all the parameters for specific cell types/samples.

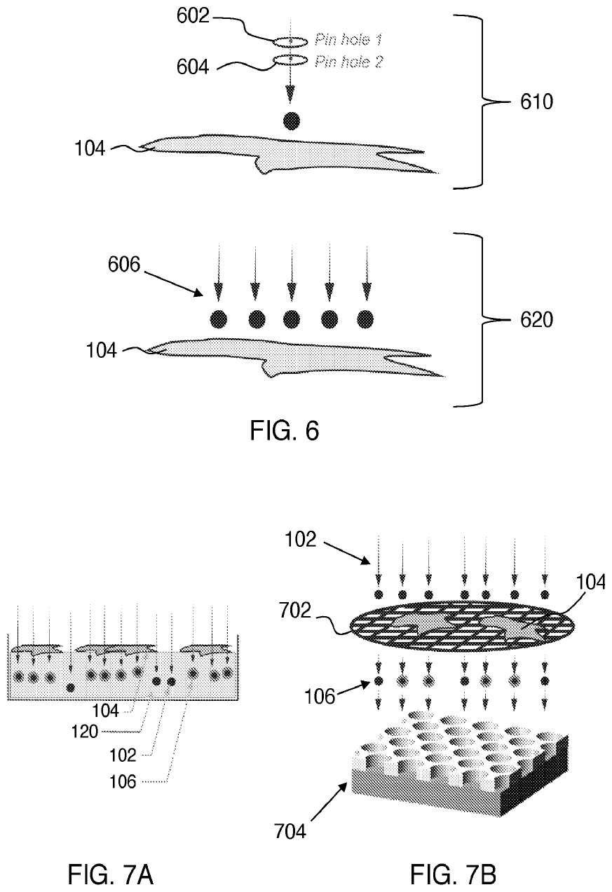

FIG. 6 shows two exemplary approaches for launching nanoparticles. In the scheme of 610, nanoparticles pass through pinholes 602 and 604 before passing through cell 104. This approach is preferred when it is desired to probe specific cell features by aiming nanoparticles at them. In the scheme of 620, the cell is flooded over a wide area with nanoparticles 606. This approach is preferred when it is desired to simultaneously sample from all or most of the cell.

FIGS. 7A-B shows two exemplary approaches for capturing nanoparticles after they have passed through the cell. The approach of FIG. 7A is use of hydrogel 120 as described above. The approach of FIG. 7B is use of a sample holder 702 to hold cells 104 above an array of two or more reaction wells 704. For example, sample holder 702 can be an electron microscope sample grid. The approach of FIG. 7B allows one to proceed with biological and/or chemical characterization of the cell contents on the nanoparticles directly in the reaction wells. Preferably, the spatial resolution of this sampling scheme is preserved by making the array of reaction wells have high resolution (e.g., 1 μm or better, where lower is better).

1. A method of sampling a biological cell, the method comprising:
firing one or more nanoparticles through one or more biological cells;
collecting nanoparticles that have passed through the one or more biological cells such that spatial registration between collected nanoparticles and corresponding biological cells is preserved; and
characterizing cell contents present on surfaces of the collected nanoparticles.

2. The method of claim 1, wherein the one or more biological cells remain intact at a rate of 90% or more after the one or more nanoparticles pass through them.

3. The method of claim 1, wherein the one or more biological cells are in a sample selected from the group consisting of: isolated cell samples, tissue slice samples and organoid samples.

4. The method of claim 1, wherein a temporal resolution of the characterizing cell contents is 1 μs or better.

5. The method of claim 1, wherein a speed range of the one or more nanoparticles is from 200 m/s to 3.5 km/s.

6. The method of claim 1, wherein a size range of the one or more nanoparticles is from 10 nm to 4 μm.

7. The method of claim 1, wherein the collecting nanoparticles that have passed through the one or more biological cells comprises passing the nanoparticles to a characterizing instrument, wherein the characterizing instrument is selected from the group of: electron microscopes, mass spectrometry instruments, sequencing instruments and optical spectroscopy instruments.

8. The method of claim 1, wherein a propulsion mechanism for the firing one or more nanoparticles through one or more biological cells is compressed gas.

9. The method of claim 1, wherein a propulsion mechanism for the firing one or more nanoparticles through one or more biological cells is laser-induced projectile formation.

10. The method of claim 1, wherein a composition of the one or more nanoparticles is selected from the group consisting of: gold, tungsten and iron.

11. The method of claim 1, wherein the one or more nanoparticles have a surface functionalization binding selected from the group consisting of: oligo-DNA binding, antibody-antigen binding, biotin binding, peptide binding, nickel-nitrilotriacetic acid binding, polyethylene glycol binding, and click chemistry binding.

12. The method of claim 1, wherein a shape of the one or more nanoparticles is selected from the group consisting of: pyramids, spheres, rods and stars.

13. The method of claim 1, wherein the collecting nanoparticles that have passed through the one or more biological cells comprises capturing the nanoparticles in a uniform hydrogel matrix.

14. The method of claim 1, wherein the collecting nanoparticles that have passed through the one or more biological cells comprises capturing the nanoparticles in an array of two or more reaction wells, wherein a spatial resolution of the array of two or more reaction wells is 1 μm or better.

15. The method of claim 14, wherein the one or more biological cells are supported by a sample holder disposed above the array of two or more reaction wells, wherein the sample holder includes an electron microscope grid.
